SEQUENCE LISTING (30 sequences)

SEQ ID NO: 1 - protein, 7 aa, synthetic construct
TVRTSAD

SEQ ID NO: 2 - protein, 7 aa, synthetic construct
NWGDRIL

SEQ ID NO: 3 - protein, 7 aa, synthetic construct
NWGKPIK

SEQ ID NO: 4 - protein, 7 aa, synthetic construct
SGVKSAF

SEQ ID NO: 5 - protein, 7 aa, synthetic construct
GVKSYNE

SEQ ID NO: 6 - protein, 7 aa, synthetic construct
IGKTNTL

SEQ ID NO: 7 - protein, 7 aa, synthetic construct
IGNSNTL

SEQ ID NO: 8 - protein, 7 aa, synthetic construct
IGNTIPV

SEQ ID NO: 9 - protein, 7 aa, synthetic construct
LYANSPF

SEQ ID NO: 10 - protein, 9 aa, synthetic construct
CTVRTSADC

SEQ ID NO: 11 - protein, 9 aa, synthetic construct
CNWGDRILC

SEQ ID NO: 12 - protein, 9 aa, synthetic construct
CNWGKPIKC

SEQ ID NO: 13 - protein, 9 aa, synthetic construct
CSGVKSAFC

SEQ ID NO: 14 - protein, 9 aa, synthetic construct
CGVKSYNEC

SEQ ID NO: 15 - protein, 9 aa, synthetic construct
CIGKTNTLC

SEQ ID NO: 16 - protein, 9 aa, synthetic construct
CIGNSNTLC

SEQ ID NO: 17 - protein, 9 aa, synthetic construct
CIGNTIPVC

SEQ ID NO: 18 - protein, 9 aa, synthetic construct
CLYANSPFC

SEQ ID NO: 19 - protein, 7 aa, synthetic construct
WNYPFRL

SEQ ID NO: 20 - protein, 7 aa, synthetic construct
SNTSYVN

SEQ ID NO: 21 - protein, 7 aa, synthetic construct
SFSYTSG

SEQ ID NO: 22 - protein, 7 aa, synthetic construct
WSPAPMS

SEQ ID NO: 23 - protein, 7 aa, synthetic construct
TREHPAQ

SEQ ID NO: 24 - protein, 7 aa, synthetic construct
ARIIDNA

SEQ ID NO: 25 - protein, 9 aa, synthetic construct
CWNYPFRLC

SEQ ID NO: 26 - protein, 9 aa, synthetic construct
CSNTSYVNC

SEQ ID NO: 27 - protein, 9 aa, synthetic construct
CSFSYTSGC

SEQ ID NO: 28 - protein, 9 aa, synthetic construct
CWSPAPMSC

SEQ ID NO: 29 - protein, 9 aa, synthetic construct
CTREHPAQC

SEQ ID NO: 30 - protein, 9 aa, synthetic construct
CARIIDNAC

(19) United States
(12) Patent Application Publication
(10) Pub. No.: US 2025/0057990 A1
(43) Pub. Date: Feb. 20, 2025

(54) TISSUE-SPECIFIC MANGANESE BASED MRI CONTRAST AGENTS

(71) Applicant: CASE WESTERN RESERVE UNIVERSITY, Cleveland, OH (US)

(72) Inventors: Zheng-Rong Lu, Cleveland, OH (US); Jing-Can Qin, Cleveland, OH (US); Ryan Hall, Cleveland, OH (US)

(21) Appl. No.: 18/721,744

(22) PCT Filed: Dec. 20, 2022
(86) PCT No.: PCT/US2022/081985

§ 371 Date: Jun. 19, 2024

Related U.S. Application Data
(60) Provisional application No. 63/291,631, filed on Dec. 20, 2021.

(51) Int. Cl. A61K 49/10; A61K 49/14; A61K 49/16

(52) U.S. Cl. CPC A61K 49/106; A61K 49/14; A61K 49/16

(57) ABSTRACT

A tissue-specific manganese based magnetic resonance imaging (MRI) agent includes the formula: wherein T includes at least one cell specific or tissue specific targeting moiety; C includes at least one pyclen based chelating agent complexed with a Mn ion, and L includes at least one optional linker that covalently links the at least one targeting moiety to the at least one chelating agent.

### RELATED APPLICATION

This application claims priority from U.S. Provisional Application No. 63/291,631, filed Dec. 20, 2021, the subject matter of which is incorporated herein by reference in its entirety.

### GOVERNMENT FUNDING

This invention was made with government support under CA211762 awarded by the National Institutes of Health. The government has certain rights in the invention.

### SEQUENCE LISTING

The information recorded in electronic form submitted under Rule 13ter.1(A) is identical to the sequence listing contained in the International Application. The sequence does not go beyond that disclosed in the International Application.

### BACKGROUND

Magnetic resonance imaging (MRI) is an increasingly attractive imaging modality in clinical practice due to its unparalleled combination of high soft tissue contrast, high resolution, and selection of imaging sequences to produce a variety of anatomical information. This is especially true for imaging of the liver, where diagnosis of liver diseases and focal liver lesions with high-resolution imaging techniques has seen a drastic increase in recent years. Currently, the only contrast agents for clinical liver MRI are the gadolinium (Gd(III))-based contrast agents (GBCAs) gadoxetate (Gd-EOB-DTPA; EOVIST) and gadobenate (Gd-BOPTA; MULTIHANCE), which utilize hydrophobic ligands for selective accumulation into hepatocytes via the organic anion transporting polypeptide 1 (OATP1) transporter. While these GBCAs produce excellent T₁-weighted (T₁w) contrast, their linear chelation structures suffer from poor kinetic stability to release toxic free Gd(III) ions via transmetallation with endogenous metal ions, such as Ca²⁺, Cu²⁺, and Zn²⁺. Long-term retention of Gd(III) in tissues, including the brain, from the linear GBCAs has raised serious safety concerns, and some of them have been prohibited for clinical use.

### SUMMARY

Embodiments described herein relate to tissue-specific manganese (Mn) based magnetic resonance imaging (MRI) contrast agents and their use in detecting, monitoring, and/or imaging organs and/or tissue of a subject.

In some embodiments, the Mn based MRI imaging agent can have the following formula:

T-L-C
wherein T includes at least one cell specific or tissue specific targeting moiety; C includes at least one pyclen based chelating agent complexed with a Mn ion, and L includes at least one optional linker that covalently links the at least one targeting moiety to the at least one chelating agent.

In some embodiments, the pyclen based chelating agent is selected from pyclen diacetate, pyclen triacetate, and derivatives thereof. For example, the pyclen based chelating agent can include at least one of pyclen-3,9-diacetate, pyclen-3,6-diacetate, or pyclen-3,6,9-triacetate.

In other embodiments, the tissue-specific Mn based MRI contrast agent can include the formula:

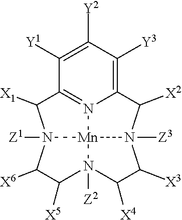

or a pharmaceutically acceptable salt thereof,
wherein:
X¹, X², X³, X⁴, X⁵, and X⁶ are each independently H, -L¹-T¹, or -T¹;
Y¹, Y², and Y³ are each independently H, -L¹-T¹, or -T¹;
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is a cell specific or tissue specific targeting moiety; and where at least one of X¹, X², X³, X⁴, X⁵, X⁶, Y¹, Y², Y³, Z¹, Z², or Z³ is -L¹-T¹ or -T¹.

In other embodiments, the tissue-specific Mn based MRI contrast agent can include the formula:

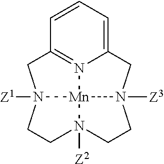

or a pharmaceutically acceptable salt thereof,
wherein:
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is a cell specific or tissue specific targeting moiety; and where at least one of Z¹, Z², or Z³ is -L-T or -T.

In other embodiments, the tissue-specific Mn based MRI contrast agent can include the formula:

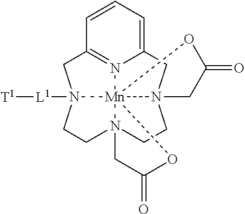

or a pharmaceutically acceptable salt thereof;
wherein L¹ is an optional linker and T¹ is a cell specific or tissue specific targeting moiety.

In some embodiments, the at least one targeting moiety includes a small molecule, peptide, antibody, or aptamer.

In some embodiments, the targeting moiety binds to and/or targets hepatocytes and/or liver tissue of a subject. For example, the targeting moiety can include an ethoxybenzyl group.

In some embodiments, a tissue-specific Mn based MRI contrast agent that binds to and/or targets hepatocytes and/or liver tissue of a subject can have the following formula:

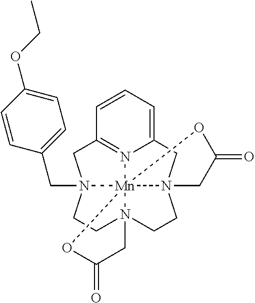

or a pharmaceutically acceptable salt thereof.

In other embodiments, the targeting moiety can bind a cancer cell antigen. The cancer cell antigen can include, for example, at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), extracellular domain B of fibronectin (EDB-FN), extracellular domain A of fibronectin (EDB-FN), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

In some embodiments, the targeting moiety can include at least one targeting peptide that includes an amino acid sequence that specifically bind to EDB-FN or EDA-FN. For example, the targeting peptide can include an amino sequence selected from TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides thereof, and retro-inverso amino acid sequences thereof.

In some embodiments, the targeting moiety is directly linked to the chelating agent.

In other embodiments, the targeting moiety is linked to the chelating agent with a linker. The linker can include a homo- or hetero-bifunctional linker. The homo or hetero-bifunctional linker can include two reactive groups are separated by at least one of an alkylene, alkenylene, alkynylene, cycloalkylenes, arylene, or araalkylene, which is optionally substituted with one or more heteroatoms. For example, the linker can include a C₁-C₆ alkylene, C₃-C₆-cycloakylene, 3 to 6 membered heterocyclylene, or arylene.

Other embodiments described herein relate to a pharmaceutical composition that includes a tissue-specific Mn based MRI contrast agent as described herein in an amount sufficient to provide a desired level of contrast and at least one pharmaceutically acceptable carrier.

Still other embodiments described herein relate to a method of detecting, monitoring, and/or imaging organs and/or tissue of a subject. The method includes administering to the subject a tissue-specific Mn based MRI contrast agent as described herein. A magnetic resonance imaging scan of the subject is performed. A measurable signal due to the agent is detected from the subject. An image from the detectable signal is generated, thereby obtaining an image of the organ or tissue of the subject. The tissue-specific Mn based MRI contrast agent can be systemically administered to the subject.

Other embodiments describe herein relate to a method of obtaining an image of a liver of a subject. The method includes administering to the subject a liver-specific Mn based MRI contrast agent of the following formula:

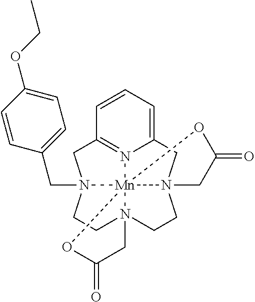

O or a pharmaceutically acceptable salt thereof. A magnetic resonance imaging scan of the subject is performed. A measurable signal due to the agent is detected from the subject. An image from the detectable signal is generated, thereby obtaining an image of the liver of the subject.

Other embodiments relate to a method of detecting, monitoring, and/or imaging cancer and/or tumors in a subject. The method includes administering to the subject a cancer and/or tumor-specific manganese based magnetic resonance imaging (MRI) agent. The agent can include the formula:

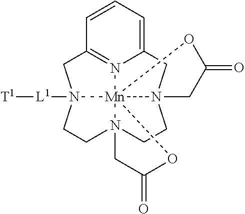

or a pharmaceutically acceptable salt thereof;
wherein L¹ is an optional linker and T¹ is a cancer and/or tumor cell specific targeting moiety. A magnetic resonance imaging scan of the subject is performed. A measurable signal due to the agent is detected from the subject. An image from the detectable signal is generated, thereby obtaining an image of the cancer and/or tumor in the subject.

In some embodiments, the cancer and/or tumor cell specific targeting moiety comprises at least one of a peptide that includes an amino acid sequence selected from TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides thereof, and retro-inverso amino acid sequences thereof.

In some embodiments, the tissue-specific Mn based MRI contrast agent is systemically administered to a subject having or suspected of having cancer.

In other embodiments, the subject can have cancer, and the tissue-specific Mn based MRI contrast agent is administered to the tissue of the subject to determine cancer aggressiveness.

### BRIEF DESCRIPTION OF THE DRAWINGS

FIG. 1 illustrates the synthesis of Mn(EOB-PC2A). Reaction conditions: (i) diethyloxalate, MeOH, RT; (ii) 1-chloromethyl-4-ethoxibenzene, ACN, K₂CO₃, refluxed; (iii) MeOH, con. H₂SO₄, refluxed; (iv) t-Bu-bromoacetate, ACN, K₂CO₃, RT; (v) TFA:DCM (1:2); (vi) MnCl₂, H₂O, pH=6.5, RT.

FIGS. 2(A-D) illustrate relaxivities, cytotoxicity and uptake in HepG2 cells of Mn(EOB-PC2A). The plots of R₁ and R₂ relaxation rates at 1.5T vs. concentration of Mn(EOB-PC2A) in (A) DPBS and (B) saline containing 4.5% human serum albumin. (C) Cell viability analysis of Mn(EOB-PC2A) in HepG2 hepatoma cells. (D) Intracellular concentration of manganese in HepG2 cells after treatment with 2 mM of Mn(EOB-PC2A) compared with untreated cells.

FIGS. 3(A-E) illustrate representative T₁-weighted axial spin-echo images (A) acquired before and at 15 minutes, 30 minutes, and 24 hours after intravenous administration of Mn(EOB-PC2A) (0.060 mmol/kg). The normalized SNR (B) and normalized CNR (C) of the liver at 15 and 30 minutes post-injection. The comparison of SNR (D) and CNR (E) of the liver before and at 24 hours after administration. (**p<0.01, ns: not significant)

FIGS. 4(A-B) illustrate representative maximum intensity projections of the 3D FLASH images (A) acquired before and at 15 minutes, 30 minutes, and 24 hours after administration of Mn(EOB-PC2A) (0.060 mmol/kg). (B) The change in SNR (delta SNR) in various tissues relative to the pre-injection baseline. (*p<0.05; **p<0.01; ***p<0.001; ns: not significant)

FIG. 5 illustrates chemical structures of liver-targeted MRI contrast agents (top row) and pyclen diacetate MBCAs (bottom row), including the liver-targeted EOB-conjugate reported in this study, Mn(EOB-PC2A).

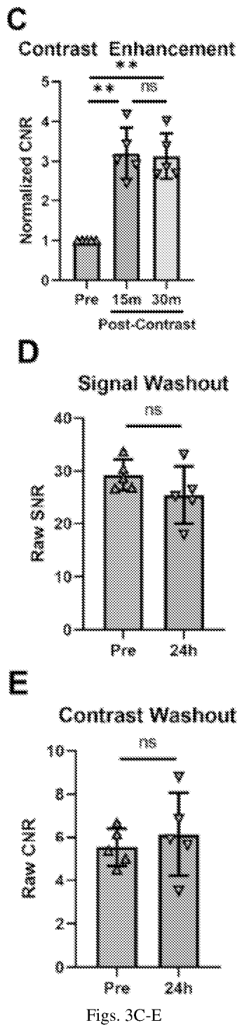

FIG. 6 illustrates a full synthesis scheme for Mn(EOB-PC2A).

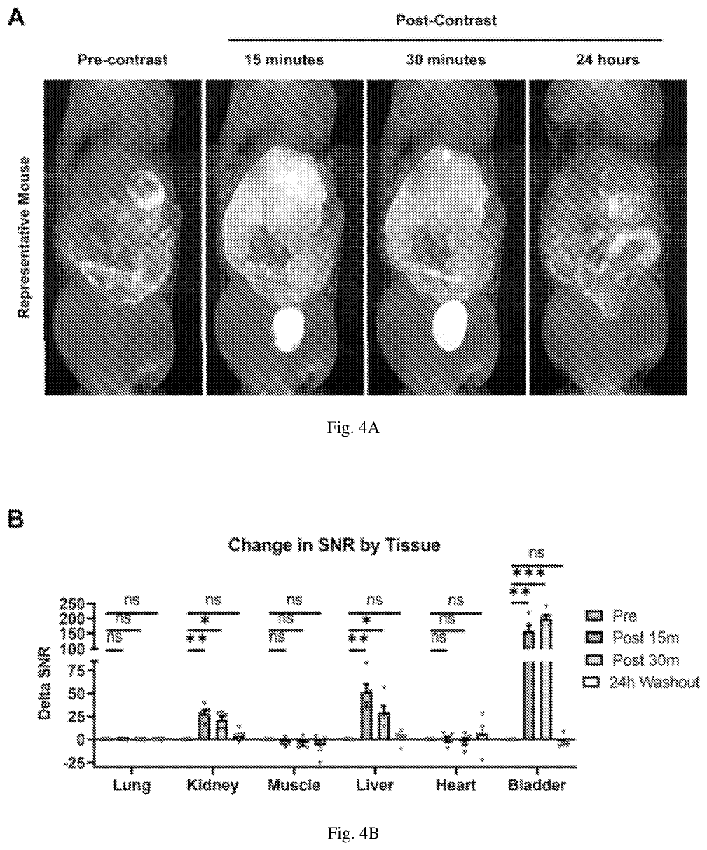

FIG. 7 illustrates relaxation rate measurements and linear estimation of the r₁ relaxivity of Mn(EOB-PC2A) at 3T in phantoms prepared in DPBS.

FIG. 8 illustrates cell viability analysis at physiologically relevant concentrations of Mn(EOB-PC2A) in DU145 prostate cancer cells.

FIG. 9 illustrates T₁-weighted axial fast spin-echo acquisitions of all mice used in the study before and at 15 minutes, 30 minutes, and 24 hours after administration of 0.060 mmol/kg Mn(EOB-PC2A). The slice shown is a representative slice containing the liver to demonstrate the robust liver enhancement and efficient 24-hour washout.

FIGS. 10(A-B) illustrate SNR and CNR analysis of the 24-hour washout of Mn(EOB-PC2A) on a mouse-by-mouse basis. Averages demonstrate no significant change from baseline.

FIG. 11 illustrates 3D FLASH acquisitions of all mice used in the study before and at 15 minutes, 30 minutes, and 24 hours after administration of 0.060 mmol/kg Mn(EOB-PC2A). The coronal slice shown is a representative slice containing the liver to demonstrate the robust liver enhancement and efficient 24-hour washout.

FIG. 12 illustrates the maximum intensity projections of the raw 3D FLASH acquisitions and corresponding subtraction images of all mice used in the study before and at 15 minutes, 30 minutes, and 24 hours after administration of 0.060 mmol/kg Mn(EOB-PC2A).

FIG. 13 illustrates the Synthesis of ZD2-(Mn-PCTA-MA) conjugates as targeted Mn(II)-based MRI contrast agents.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

The terms “comprise,” “comprising,” “include,” “including,” “have,” and “having” are used in the inclusive, open sense, meaning that additional elements may be included. The terms “such as”, “e.g.,” as used herein are non-limiting and are for illustrative purposes only. “Including” and “including but not limited to” are used interchangeably.

The term “or” as used herein should be understood to mean “and/or”, unless the context clearly indicates otherwise.

As used herein, the term “subject” can refer to any animal including, but not limited to, humans and non-human animals (e.g., rodents, arthropods, insects, fish (e.g., zebrafish)), non-human primates, ovines, bovines, ruminants, lagomorphs, porcines, caprines, equines, or canines felines, aves, etc.).

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

As used herein, the term “small molecule” can refer to lipids, carbohydrates, polynucleotides, polypeptides, or any other organic or inorganic molecules.

As used herein, the term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polypeptide means at least about three sequential amino acid residues of the polypeptide. It is understood that a portion of a polypeptide may include every amino acid residue of the polypeptide.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

As used herein, the term “targeting moiety” can refer to a molecule or molecules that are able to bind to and complex with a biomarker. The term can also refer to a functional group that serves to target or direct a therapeutic agent to a particular location, cell type, diseased tissue, or association. In general, a “targeting moiety” can be directed against a biomarker.

The term “neoplastic disorder” can refer to a disease state in a subject in which there are cells and/or tissues which proliferate abnormally. Neoplastic disorders can include, but are not limited to, cancers, sarcomas, tumors, leukemias, lymphomas, and the like.

The term “neoplastic cell” can refer to a cell that shows aberrant cell growth, such as increased, uncontrolled cell growth. A neoplastic cell can be a hyperplastic cell, a cell from a cell line that shows a lack of contact inhibition when grown in vitro, a tumor cell, or a cancer cell that is capable of metastasis in vivo. Alternatively, a neoplastic cell can be termed a “cancer cell.” Non-limiting examples of cancer cells can include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, pancreatic carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

Embodiments described herein relate to tissue-specific manganese (Mn) based magnetic resonance imaging (MRI) contrast agents and their use in detecting, monitoring, and/or imaging organs and/or tissue of a subject. In some embodiments, the tissue or organ to be imaged can include liver tissue, and the tissue-specific Mn based MRI contrast agent can specifically bind to or target liver cells, such as hepatocytes, or liver tissue for imaging of liver diseases including chronic liver diseases, and tumors of the liver. In other embodiments, the tissue can include cancer or tumor tissue and the tissue-specific Mn based MRI contrast agent can specifically bind to or target cancer cells or tumors. The cancer cell or tumor-specific Mn based MRI contrast agent can be used for detecting, monitoring, and/or imaging cancer distribution and/or location and/or cancer cell metastasis, migration, and/or invasion in a subject, detecting and/or monitoring cancer cell aggressiveness and/or malignancy in a subject, and/or determining and/or monitoring the efficacy of a cancer therapeutic and/or cancer therapy administered to a subject in need thereof.

In some embodiments, the tissue-specific Mn based MRI contrast agent can have the following formula:

T-L-C
wherein T includes at least one cell specific or tissue specific targeting moiety; C includes at least one pyclen based chelating agent complexed with a Mn ion, and L includes at least one optional linker that covalently links the at least one targeting moiety to the at least one chelating agent.

In some embodiments, the pyclen based chelating agent is selected from pyclen diacetate, pyclen triacetate, and derivatives thereof. For example, the pyclen based chelating agent can include at least one of pyclen-3,9-diacetate, pyclen-3,6-diacetate, or pyclen-3,6,9-triacetate.

In other embodiments, the tissue-specific Mn based MRI contrast agent can include the formula:

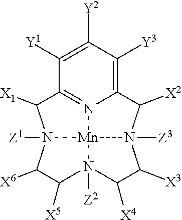

or a pharmaceutically acceptable salt thereof,
wherein:
X¹, X², X³, X⁴, X⁵, and X⁶ are each independently H, -L¹-T¹, or -T¹;
Y¹, Y², and Y³ are each independently H, -L¹-T¹, or -T¹;
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is a cell specific or tissue specific targeting moiety; and where at least one of X¹, X², X³, X⁴, X⁵, X⁶, Y¹, Y², Y³, Z¹, Z², or Z³ is -L¹-T¹ or -T¹.

In other embodiments, the tissue-specific Mn based MRI contrast agent can include the formula:

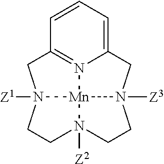

or a pharmaceutically acceptable salt thereof,
wherein:
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is a cell specific or tissue specific targeting moiety; and where at least one of Z¹, Z², or Z³ is -L-T or -T.

In other embodiments, the tissue-specific Mn based MRI contrast agent can include the formula:

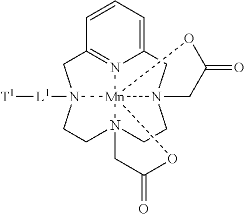

or a pharmaceutically acceptable salt thereof;
wherein L¹ is an optional linker and T¹ is a cell specific or tissue specific targeting moiety.

The targeting moieties are configured to specifically bind to and/or target molecules of a cell, tissue, and/or disease site of interest in a subject. The targeting moieties can also be capable of targeting and/or adhering the tissue-specific Mn based MRI contrast agent to the targeted cell, tissue, and/or disease site of interest. The targeting moiety can include any molecule, or complex of molecules, which is/are capable of interacting with a cell surface or extracellular molecule or biomarker of a cell. The cell surface molecule can include, for example, a cellular protease, a kinase, a protein, a cell surface receptor, a lipid, and/or fatty acid.

In certain embodiments, the targeting moiety specifically binds a cell surface molecule of a target cell. As used herein, a targeting moiety “specifically binds” to a target molecule if it binds to or associates with the target molecule with an affinity or Ka (that is, an equilibrium association constant of a particular binding interaction with units of 1/M) of, for example, greater than or equal to about 10⁵ M⁻¹. In certain embodiments, the first molecule binds to the second molecule with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the first molecule for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In some embodiments, the targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide Fv fragments, scFv tandems ((scFv)2 fragments), diabodies, tribodies or tetrabodies, which typically are covalently linked or otherwise stabilized (i.e., leucine zipper or helix stabilized) scFv fragments; and receptor molecules, which naturally interact with a desired target molecule.

Preparation of antibodies may be accomplished by any number of well-known methods for generating antibodies. These methods typically include the step of immunization of animals, typically mice, with a desired immunogen (e.g., a desired target molecule or fragment thereof). Once the mice have been immunized and boosted one or more times with the desired immunogen(s), antibody-producing hybridomas may be prepared and screened according to well-known methods. See, for example, Kuby, Janis, Immunology, Third Edition, pp. 131-139, W.H. Freeman & Co. (1997), for a general overview of monoclonal antibody production, that portion of which is incorporated herein by reference.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

The immunogens used to prepare targeting moieties having a desired specificity will generally be the target molecule, or a fragment or derivative thereof. Such immunogens may be isolated from a source where they are naturally occurring or may be synthesized using methods known in the art. For example, peptide chains may be synthesized by 1-ethyl-3-[dimethylaminopropyl]carbodiimide (EDC)-catalyzed condensation of amine and carboxyl groups. In certain embodiments, the immunogen may be linked to a carrier bead or protein. For example, the carrier may be a functionalized bead such as SASRIN resin commercially available from Bachem, King of Prussia, PA. or a protein such as keyhole limpet hemocyanin (KLH) or bovine serum albumin (BSA). The immunogen may be attached directly to the carrier or may be associated with the carrier via a linker, such as a non-immunogenic synthetic linker (for example, a polyethylene glycol (PEG) residue, amino caproic acid or derivatives thereof) or a random, or semi-random polypeptide.

In certain embodiments, it may be desirable to mutate a binding region of the polypeptide targeting moiety and select for a targeting moiety with superior binding characteristics as compared to the un-mutated targeting moiety. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the targeting moiety may be modified to make them more resistant to cleavage by proteases. For example, the stability of a targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, antibodies or variants thereof may be modified to make them less immunogenic when administered to a subject. For example, if the subject is human, the antibody may be “humanized”; where the complimentarily determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete.

In certain embodiments, a targeting moiety as described herein may comprise a homing peptide, which selectively directs the tissue-specific Mn based MRI contrast agent to a targeted cell. Homing peptides for a targeted cell can be identified using various methods well known in the art. Many laboratories have identified the homing peptides that are selective for cells of the vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996 Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,6999; 6,068,829; 6,174,687; 6,180,084; 6,232,287; 6,296,832; 6,303,573; and 6,306,365.

Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or cell surface receptors (see, e.g., Smith, et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol. 124:373-380), and to the human urokinase receptor (Goodson, et al., 1994, Proc. Natl. Acad. Sci., USA 91:7129-7133).

In certain embodiments, the targeting moiety may comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the targeting moiety may comprise a ligand molecule, including, for example, ligands which naturally recognize a specific desired receptor of a target cell. Such ligand molecules include ligands that have been modified to increase their specificity of interaction with a target receptor, ligands that have been modified to interact with a desired receptor not naturally recognized by the ligand, and fragments of such ligands.

In other embodiments, the targeting moiety can include a molecule that promotes selective uptake of the Mn based MRI contrast agent by a specific cell. For example, the targeting moiety can include ethoxy benzyl, which can promote uptake of the Mn based MRI contrast agent by hepatocytes or other cells of the liver.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein, which is involved in binding other proteins, peptidomimetic compounds can be generated that mimic those residues, which facilitate the interaction. Such mimetics may then be used as a targeting moiety to deliver the nanobubble to a target cell. For instance, non-hydrolyzable peptide analogs of such resides can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Cummun 126:419; and Dann et al., 1986, Biochem Biophys Res Commun 134:71).

In some embodiments, the targeting moiety can bind to or specifically bind to a cancer antigen. The cancer cell antigen can include at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), extracellular domain B of fibronectin (EDB-FN), extracellular domain A of fibronectin (EDB-FN, fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

Non-limiting examples of antibodies that specifically bind to tumor antigens which may be used as a targeting moiety include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sotituzumab, Catumaxomab, Ertumaxomab, Depatuxizumab, Ontuxizumab, Blontuvetmab, Tamtuvetmab, or a tumor antigen-binding variant thereof. As used herein, “variant” is meant the antibody specifically binds to the particular antigen (e.g., HER2 for trastuzumab) but has fewer or more amino acids than the parental antibody (e.g., is a fragment (e.g., scFv) of the parental antibody), has one or more amino acid substitutions relative to the parental antibody, or a combination thereof.

In some embodiments, the targeting moiety can include a targeting peptide that can specifically bind to EDB-FN. Targeting peptides that specifically bind EDB-FN can include linear peptides having the amino acid sequences of TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), cyclic peptides having the amino acid sequences of CTVRTSADC (SEQ ID NO: 10), CNWGDRILC (SEQ ID NO: 11), CNWGKPIKC (SEQ ID NO: 12), CSGVKSAFC (SEQ ID NO: 13), CGVKSYNEC (SEQ ID NO: 14), CIGKTNTLC (SEQ ID NO: 15), CIGNSNTLC (SEQ ID NO: 16), CIGNTIPVC (SEQ ID NO: 17), or CLYANSPFC (SEQ ID NO: 18), linear peptides with cysteine linkers, or retro-inverso peptides having a retro-inverso amino acid sequence of the linear peptides thereof.

In other embodiments, the targeting moiety can include a targeting peptide that specifically binds to EDA-FN. Targeting peptides that specifically bind EDA-FN can include linear peptides having the amino acid sequences of WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides having the amino acid sequences of CWNYPFRLC (SEQ ID NO: 25), CSNTSYVNC (SEQ ID NO: 26), CSFSYTSGC (SEQ ID NO: 27), CWSPAPMSC (SEQ ID NO: 28), CTREHPAQC (SEQ ID NO: 29), or CARIIDNAC (SEQ ID NO: 30), linear peptides with cysteine linkers, or retro-inverso peptides having a retro-inverso amino acid sequence of the linear peptides thereof.

The targeting peptides can be subject to various changes, substitutions, insertions, and deletions where such changes provide for certain advantages in its use. In this regard, targeting peptides that bind to and/or complex with EDB-FN and/or EDA-FN can be substantially homologous with, rather than be identical to, the sequence of a recited peptide where one or more changes are made and it retains the ability to function as specifically binding to and/or complexing with EDB-FN and/or EDA-FN.

The targeting peptides can be in any of a variety of forms of polypeptide derivatives, that include amides, conjugates with proteins, cyclized polypeptides, polymerized polypeptides, retro-inverso peptides, analogs, fragments, chemically modified polypeptides, and the like derivatives.

Retro-inverso peptides are linear peptides whose amino acid sequence is reversed and the α-center chirality of the amino acid subunits is inverted as well. These types of peptides are designed by including D-amino acids in the reverse sequence to help maintain side chain topology similar to that of the original L-amino acid peptide and make them more resistant to proteolytic degradation. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used. Furthermore, the insertion of D-amino acids in selected sequence regions as sequence blocks containing only D-amino acids or in-between L-amino acids allows the design of targeting peptides that are bioactive and possess increased bioavailability in addition to being resistant to proteolysis. Furthermore, if properly designed, retro-inverso peptides can have binding characteristics similar to L-peptides.

The term “analog” includes any peptide having an amino acid residue sequence substantially identical to a sequence specifically shown herein in which one or more residues have been conservatively substituted with a functionally similar residue and that specifically binds to and/or complexes with EDB-FN and/or EDA-FN as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Peptides described herein also include any peptide having one or more additions and/or deletions or residues relative to the sequence of a peptide whose sequence is shown herein, so long as the requisite binding specificity or activity is maintained.

The term “fragment” refers to any subject peptide having an amino acid residue sequence shorter than that of a polypeptide whose amino acid residue sequence is shown herein.

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

The targeting peptides can be synthesized by any of the techniques that are known to those skilled in the polypeptide art, including recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

Furthermore, the targeting peptides described herein can be used as a starting point to develop higher affinity small molecules, peptides, antibodies, and/or antibody fragments with similar ligand binding capabilities. The development and screening of small molecules from pharmacophores of the peptides using, for example, in silico screening, can be readily performed, and the binding affinity of such identified molecules can be readily screened against targeting peptides using assays described herein to select small molecule agents.

Additional residues may also be added at either terminus of a peptide for the purpose of conveniently linking the targeting peptide to the optional linker or pyclen based chelating agent.

The targeting moiety can be linked or conjugated directly to the pyclen based chelating agent or be linked to the pyclen based chelating agent via the linker. The linker can include a homo- or hetero-bifunctional linker. The homo or hetero-bifunctional linker can include two reactive groups are separated by at least one of an alkylene, alkenylene, alkynylene, cycloalkylenes, arylene, or araalkylene, which is optionally substituted with one or more heteroatoms so that ideally the spatial configuration of the targeting moiety is not influenced by the presence of the chelating agent. For example, the linker can include a C₁-C₆ alkylene, C₃-C₆-cycloakylene, 3 to 6 membered heterocyclylene, or arylene.

The reactive moieties in the bifunctional linker, which may be the same or, different, can be capable of reaction with a functional groups present in the chelating agent and targeting moiety, such as carboxylic groups, sulphydryl groups, and amino groups. Examples of functional groups capable of reaction with carboxylic groups include diazo compounds such as diazoacetate esters and diazoacetamides, which-react with high specificity to generate ester groups. Carboxylic acid modifying reagents, such as carbodiimides, e.g. 1-cyclohexyl-3-(2-morpholinyl-4-ethyl)carbodiimide (CMC) and 1-ethyl-3-(3-dimethylaminopropyl)carbodiimide (EDC), may also be usefully employed. Other useful carboxylic acid modifying reagents include isoxazolium derivatives, chloroformates, and N-carbalkoxydihydroquinoline. Examples of reactive moieties capable of reaction with sulphydryl groups include α-haloacetyl compounds and maleimide derivatives. Examples of reactive moieties capable of reaction with amino groups include alkylating and acylating agents. Representative of the alkylating agents are α-haloacetyl compounds, maleimide derivatives, reactive aryl halides and alkyl halides, α-haloalkyl ethers, aldehydes and ketones capable of Schiffs base formation with amino groups (the adducts formed usually being stabilized through reduction to give a stable amine), epoxide derivatives, such as epichlorohydrin and bisoxiranes. Examples of acylating agents include isocyanates and isothiocyanates, acid anhydrides, acid halides, active esters, and those useful reagents for amide bond formation widely known and conventionally used for peptide syntheses.

In some embodiments, useful reactive groups are present on the targeting peptides based on the particular amino acids present, and additional groups can be designed. It will be evident to those skilled in the art that a variety of bifunctional or polyfunctional reagents, both homo- and hetero-functional (such as those described in the catalog of the Pierce Chemical Co., Rockford, Ill.), can be employed as a reactive group.

Other types of binding chemistries are also available. For example, methods for conjugating polysaccharides to peptides are exemplified by, but not limited to coupling via alpha- or epsilon-amino groups to NaIO₄-activated oligosaccharide (Bocher et al., J. Immunol. Methods 27, 191-202 (1997)), using squaric acid diester (1,2-diethoxycyclobutene-3,4-dione) as a coupling reagent (Tietze et al. Bioconjug Chem. 2:148-153 (1991)), coupling via a peptide binder wherein the polysaccharide has a reducing terminal and is free of carboxyl groups (U.S. Pat. No. 5,342,770), and coupling with a synthetic peptide carrier derived from human heat shock protein hsp65 (U.S. Pat. No. 5,736,146). Further methods for conjugating polysaccharides, proteins, and lipids to peptides are described by U.S. Pat. No. 7,666,624.

In some embodiments, the linker is a non-peptide linker. The non-peptide linker can be a non-peptide aliphatic, heteroaliphatic, cyclic, and/or heterocyclic linker. The non-peptide linker can include, for example, an alkylene, alkylene oxide, arylene, or alkylenearylene linker that covalently links the peptide and contrast agent.

The linker may also contain more than two functional groups. In particular when a single linker is used to bind more than one chelating agent to one or more than one targeting moiety or vice-versa, the linker may contain a multiplicity of possible binding sites or be a molecular aggregate with a multiplicity of built-in or pendant groups which bind covalently or non-covalently with the chelating agent and the targeting moiety in such a way to anchor said moieties thereto with a strength and for a time sufficient to bring the chelating agent into the cells.

In some embodiments, the nature of the linker may have a bearing on the stability of the pyclen based chelating agent and on the capability of the targeting moiety to recognize specific cells or tissue in the subject. As indicated, the linker should bind the chelating to the targeting moiety for an adequate period of time that would allow the chelating agent to be internalized by a cell, such as a hepatocyte or tumor cell, in an amount sufficient to give a clearly distinguishable contrast imaging. The linker should also bind the chelating agent to the targeting moiety in a way that would ensure an appropriate spatial conformation that allows the targeting moiety to specifically bind to cell or tissue of interest. Furthermore, the optional linker should also ensure that water molecules have access to the chelated Mn ion (as the entity of the signal will depend on the rate of exchange of the water molecule of the coordination cage with the water bulk).

When more than one molecule of linker is present, each of them can be independently selected as indicated above.

The tissue-specific Mn based MRI contrast agents described herein can be administered to the subject by, for example, systemic, topical, and/or parenteral methods of administration. These methods include, e.g., injection, infusion, deposition, implantation, or topical administration, or any other method of administration where access to the tissue by the tissue-specific Mn based MRI contrast agent is desired. In one example, administration of the tissue-specific Mn based MRI contrast agent can be by intravenous injection of the tissue-specific Mn based MRI contrast agent in the subject. Single or multiple administrations of the tissue-specific Mn based MRI contrast agent can be given. “Administered”, as used herein, means provision or delivery of a tissue-specific Mn based MRI contrast agent in an amount(s) and for a period of time(s) effective to label cancer cells in the subject.

The tissue-specific Mn based MRI contrast agents described herein can be administered to a subject in a detectable quantity of a pharmaceutical composition containing a tissue-specific Mn based MRI contrast agent or a pharmaceutically acceptable water-soluble salt thereof, to a patient.

A “detectable quantity” means that the amount of tissue-specific Mn based MRI contrast agent that is administered is sufficient to enable detection of binding or complexing of the tissue-specific Mn based MRI contrast agent to the targeted cell or tissue. An “imaging effective quantity” means that the amount of the tissue-specific Mn based MRI contrast agents that is administered is sufficient to enable imaging of binding or complexing of the tissue-specific Mn based MRI contrast agent to the targeted cell or tissue.

Formulation of the tissue-specific Mn based MRI contrast agents to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases, such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

In some embodiments, tissue-specific Mn based MRI contrast agent can be used in a method of detecting, monitoring, and/or imaging organs and/or tissue of a subject. The method includes administering to the subject the Mn based MRI contrast agent. A magnetic resonance imaging scan of the subject can be performed. A measurable signal due to the agent is detected from the subject. An image from the detectable signal is generated, thereby obtaining an image of the organ or tissue of the subject. The agent can be systemically administered to the subject.

In some embodiments, the Mn based MRI contrast agent can be used for imaging liver of a subject. In particular, the Mn based MRI contrast agents can be used for imaging of liver diseases including, but not limited to, chronic diseases and tumors of the liver. The Mn based MRI contrast agent allow, for example, the imaging of liver diseases selected from liver inflammation (hepatitis), liver cirrhosis (shrunken liver), fatty liver, autoimmune liver diseases such as autoimmune hepatitis (AIH), primarily sclerosing cholangitis (PSC) and primarily biliary cirrhosis (PBC), and iron storage disease (hemochromatosis).

The Mn based MRI contrast agent allow imaging of all primary and secondary tumors of the liver and bile ducts, for example hemangioma, hepatocellular adenoma, focal nodular hyperplasia (FNH), nodular regenerative hyperplasia (NRH), cholangioadenoma; hepatocellular carcinoma, cholangiocarcinoma, cystadenocarcinoma, angiosarcoma and hepatoblastoma, metastases of other tumors such as colorectal carcinoma; appendiceal carcinoids; mammary carcinoma, ovarian carcinoma, lung carcinoma, renal carcinoma, carcinoma of the prostate, and others.

In some embodiments, the Mn based MRI contrast agent can be used in a method of imaging liver of a subject. The method includes administering to the subject a tissue-specific Mn based MRI contrast agent of the following formula:

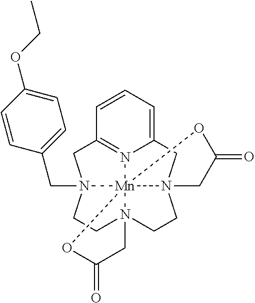

or a pharmaceutically acceptable salt thereof. A magnetic resonance imaging scan of the subject is performed. A measurable signal due to the agent is detected from the subject. An image from the detectable signal is generated, thereby obtaining an image of the liver of the subject.

In other embodiments, the tissue-specific Mn based MRI contrast agents described herein can be used in a method to detect and/or determine the presence, location, and/or distribution of cancer cells expressing EDB-FN and/or EDA-FN, in an organ, tissue, or body area of a subject. The presence, location, and/or distribution of the tissue-specific Mn based MRI contrast agent in the animal's tissue, e.g., prostate tissue, can be visualized (e.g., with an in vivo imaging modality described above). “Distribution” as used herein is the spatial property of being scattered about over an area or volume. In this case, “the distribution of cancer cells” is the spatial property of cancer cells being scattered about over an area or volume included in the animal's tissue, e.g., prostate tissue. The distribution of the tissue-specific Mn based MRI contrast agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject.

In other embodiments, the tissue-specific Mn based MRI contrast agents may be administered to a subject to assess the distribution of malignant or metastatic cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of tissue on the tumor margin that are outside the tumor edge that appear to be grossly normal but are infiltrated by dispersing tumor cells upon histological examination.

The tissue-specific Mn based MRI contrast agents specifically bind to and/or complex with EDB-FN and/or EDA-FN associated with malignant or metastatic cells can be used in intra-operative imaging techniques to guide surgical resection and eliminate the “educated guess” of the location of the tumor margin by the surgeon. Previous studies have determined that more extensive surgical resection improves patient survival. Thus, the tissue-specific Mn based MRI contrast agents that function as diagnostic molecular imaging agents have the potential to increase patient survival rates.

In some embodiments, to identify and facilitate removal of cancers cells, microscopic intra-operative imaging (IOI) techniques can be combined with systemically administered or locally administered tissue-specific Mn based MRI contrast agents described herein. The tissue-specific Mn based MRI contrast agents upon administration to the subject can target and detect and/or determine the presence, location, and/or distribution of cancer cells, i.e., cancer cells associated with EDB-FN and/or EDA-FN expression, in an organ or body area of a patient. In one example, the tissue-specific Mn based MRI contrast agents can be combined with IOI to identify malignant cells that have infiltrated and/or are beginning to infiltrate at a tumor margin. The method can be performed in real-time during surgery. The method can include local or systemic application of the tissue-specific Mn based MRI contrast agents that includes the tissue-specific Mn based MRI contrast agent. An imaging modality can then be used to detect and subsequently gather image data. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

Another embodiment described herein relates to a method of determining the aggressiveness or malignancy of cancer cells in a subject. It was found that the binding intensity of the tissue-specific Mn based MRI contrast agents to a cancer correlated with the cancer aggressiveness. Enhanced binding correlated with more aggressive cancer whereas lower or reduced binding correlated with less aggressive or benign tumors. In one example, binding of the tissue-specific Mn based MRI contrast agents to prostate tumor sections correlated with to Gleason score based on tumor aggressiveness, where enhanced binding intensity of the tissue-specific Mn based MRI contrast agents correlated to aggressive or malignant prostate cancer and which was distinguished from benign prostatic hyperplasia, which displayed lower binding intensity of the tissue-specific Mn based MRI contrast agents. The tissue-specific Mn based MRI contrast agents described herein can be used to monitor and/or compare the aggressiveness a cancer in a subject prior to administration of a cancer therapeutic or cancer therapy, during administration, or post therapeutic regimen.

Another embodiment described herein relates to a method of monitoring the efficacy of a cancer therapeutic or cancer therapy administered to a subject. The methods and the tissue-specific Mn based MRI contrast agents described herein can be used to monitor and/or compare the aggressiveness, invasion, migration, dispersal, and metastases of a cancer in a subject prior to administration of a cancer therapeutic or cancer therapy, during administration, or post therapeutic regimen.

A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The method of monitoring the efficacy of a cancer therapeutic can include the steps of administering in vivo to the animal the tissue-specific Mn based MRI contrast agents as described herein, then visualizing a distribution of the tissue-specific Mn based MRI contrast agent in the animal (e.g., with an in vivo imaging modality as described herein), and then correlating the distribution of the tissue-specific Mn based MRI contrast agent with the efficacy of the cancer therapeutic. It is contemplated that the administering step can occur before, during, and after the course of a therapeutic regimen in order to determine the efficacy of a chosen therapeutic regimen. One way to assess the efficacy of the cancer therapeutic is to compare the distribution of a tissue-specific Mn based MRI contrast agent pre and post cancer therapy.

In some embodiments, the Mn based MRI contrast agent bound to and/or complexed with the EDB-FN and/or EDA-FN is detected in the subject to detect and/or provide the aggressiveness, location and/or distribution of the cancer cells in the subject. The aggressiveness, location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the tissue-specific Mn based MRI contrast agent to the subject and detecting the Mn based MRI contrast agent bound to and/or complexed with cancer cells in the subject prior to administration of the cancer therapeutic and/or cancer therapy.

In certain embodiments, the methods and the tissue-specific Mn based MRI contrast agent described herein can be used to measure the efficacy of a therapeutic administered to a subject for treating a metastatic or aggressive cancer. In this embodiment, the tissue-specific Mn based MRI contrast agent can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the tissue-specific Mn based MRI contrast agent can be used to define the distribution of the metastatic cancer pre-operative and post-operative to determine the efficacy of the surgical resection. Optionally, the methods and tissue-specific Mn based MRI contrast agent can be used in an intra-operative surgical procedure, such as a surgical tumor resection, to more readily define and/or image the cancer cell mass or volume during the surgery.

Other embodiments relate to a method of detecting, monitoring, and/or imaging cancer and/or tumors in a subject. The method includes administering to the subject a cancer and/or tumor-specific manganese based magnetic resonance imaging (MRI) agent. The agent can include the formula:

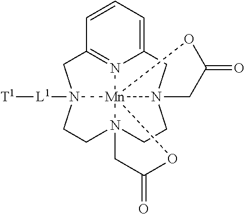

or a pharmaceutically acceptable salt thereof;
wherein L¹ is an optional linker and T¹ is a cancer and/or tumor cell specific targeting moiety. A magnetic resonance imaging scan of the subject is performed. A measurable signal due to the agent is detected from the subject. An image from the detectable signal is generated, thereby obtaining an image of the cancer and/or tumor in the subject.

In some embodiments, the cancer and/or tumor cell specific targeting moiety comprises at least one of a peptide that includes an amino acid sequence selected from TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides thereof, and retro-inverso amino acid sequences thereof.

In some embodiments, the agent is systemically administered to a subject having or suspected of having cancer.

In other embodiments, the subject can have cancer and the agent is administered to the tissue of the subject to determine cancer aggressiveness.

The following example is for the purpose of illustration only and is not intended to limit the scope of the claims, which are appended hereto.

### Example 1

This example describes the synthesis, characterization, and efficacy of a novel manganese based contrast agent (MBCA) of pyclen diacetate with an ethoxybenzyl (EOB) ligand as a liver-specific MRI contrast agent.

### Materials and Instruments for Chemistry

Unless otherwise specified, all chemicals and reagents were purchased from Sigma-Aldrich and used without further purification (St. Louis, MO, USA). ¹H NMR spectra were determined using a Bruker Avance III HD 500 NMR Spectrometer with an internal standard. Matrix-assisted laser desorption/ionization time-of-flight (MALDI-TOF) mass spectra were recorded on a Voyager DE-STR Spectrometer (PerkinElmer, Waltham, MA) in the linear mode with 2,5-dihydroxybenzoic acid as the matrix. Electrospray ionization (ESI) mass spectra were acquired using a Thermo Quest Finnigan LCQ Deca mass spectrometer system (Conquer Scientific, Poway, CA). Analytical high performance liquid chromatography (HPLC) was performed using an Agilent 1260 Infinity II LC System (Agilent, Santa Clara, CA). Preparative HPLC was performed using an Agilent 1100 Series Capillary LC System (Agilent).

Synthesis of 2,6-bis(hydroxymethyl)pyridine (Compound 1)

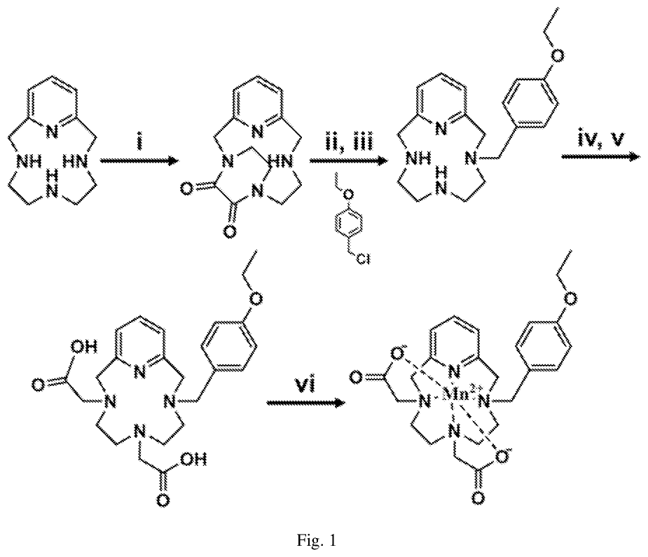

The full synthesis scheme is shown in FIG. 1. NaBH₄ (3 g, 7.91 mmol) was added to a solution of THF and CH₃OH (50 mL:10 mL) containing dimethyl 2,6-pyridinedicarboxylate (5.00 g, 25.64 mmol) at 0° C. for 0.25 h, and stirred at room temperature for 24 h. The solvent was evaporated, and the residue was redissolved in 30 mL H₂O, adjusted to pH=3 by 37% HCl. After removal of the solvents with a rotavapor, the residue was extracted with ethanol (4×80 mL), followed by removal of the ethanol and purification by silica gel column chromatography (elution: CH₃OH:CH₂Cl₂=1:7). Yield: 88%. ¹H NMR (500 MHz, DMSO-d₆). δ 7.78 (t, J=7.7 Hz, 1H), 7.32 (d, J=7.7 Hz, 2H), 5.37 (t, J=5.9 Hz, 2H). 4.52 (d, J=5.9 Hz, 4H).

### Synthesis of 2,6-bis(chloromethyl)pyridine (Compound 2)

2,6-Bis(hydroxymethyl)pyridine (3.0 g, 19 mmol) was slowly added to 20 mL SOCl₂ and refluxed for 4 h. After cooling to room temperature, excess SOCl₂ was removed by evaporation, and then 20 mL water was added. The solution was adjusted to pH=7 using saturated NaHCO₃ aqueous solution. Precipitates were filtered, washed with water (3×30 mL), and dried. Yield: 49%. ¹H NMR (500 MHz, CDCl₃). δ ppm: 7.76 (t, J=7.7 Hz, 1H), 7.44 (d, J=7.7 Hz, 2H), 4.59 (s, 4H).

### Synthesis of tritosyl-pyclen (Compound 3)

N,N′,N″-Tris(p-toluensulfonyl)diethylentriamine (5.67 g, 10 mmol) and K₂CO₃ (2.76 g, 20 mmol) were suspended in 230 mL acetonitrile, 2,6-bis(chloromethyl)pyridine (1.76 g, 10 mmol) in 85 mL acetonitrile was added dropwise with stirring within 4 h, and the mixture was then refluxed for 18 h. After filtration, the filtrate was removed to obtain a yellow solid. The product was recrystallized using acetonitrile to give a white solid. Yield: 89%. ¹H NMR (500 MHz, CDCl₃). δ ppm: 7.68 (t, J=7.6 Hz, 1H), 7.66 (d, J=7.6 Hz, 4H), 7.59 (d, J=7.6 Hz, 2H), 7.37 (d, J=7.4 Hz, 2H), 7.28 (d, J=7.4 Hz, 4H), 7.21 (d, J=7.4 Hz, 2H), 4.22 (bs, 4H), 3.24 (t, J=7.4 Hz, 4H), 2.68 (bs, 4H), 2.38 (s, 6H), 2.34 (s, 3H).

### Synthesis of pyclen (Compound 4)

Tritosyl-pyclen (3 g, 4.48 mmol) was added to 20 mL H₂SO₄ (98%) and stirred for 30 minutes at 160° C. After cooling to room temperature, the reaction mixture was carefully added to 40 mL water with stirring and extracted with CH₂Cl₂ (3×30 mL), and then the aqueous phase was adjusted to pH=14 using 30% NaOH aqueous solution and extracted with CH₂Cl₂ (3×50 mL). The organic phase was dried with anhydrous MgSO₄, filtered and evaporated to obtained a white solid. Yield: 65%. ¹H NMR (500 MHz, CDCl₃). δ ppm: 7.53 (t, J=7.6 Hz, 1H), 7.05 (d, J=7.6 Hz, 2H), 3.98 (s, 4H), 2.97 (s, 3H) 2.71-2.68 (m, 4H), 2.27-2.24 (m, 4H).

### Synthesis of pyclen oxalate (Compound 5)

A solution of diethyloxalate (0.421 g, 2.85 mmol) in 10 mL MeOH was added to a solution of pyclen (0.588 g, 2.85 mmol) 20 mL in MeOH. The reaction mixture was stirred at room temperature overnight and then concentrated. The residue was taken in dichloromethane, filtered and concentrated to remove the unreacted pyclen. The crude product was purified by silica gel column chromatography (elution:methanol). Yield: 55%. ¹H NMR (500 MHz, CDCl₃). δ ppm: δ 7.52 (t, J=7.7 Hz, 1H), 7.02 (d, J=7.9 Hz, 1H), 6.93 (d, J=7.5 Hz, 1H), 5.59 (d, J=16.2 Hz, 1H), 4.62 (m 1H), 4.08 (d, J=16.6 Hz, 1H), 3.95 (d, J=17.3 Hz, 1H), 3.77 (m, 1H), 3.70 (d, J=17.3 Hz, 1H), 3.5 (m, 1H), 3.24 (m, 1H), 3.13 (m, 1H), 3.01 (dt, J=12.2 Hz, J=3.2 Hz, 1H), 2.83 (dt, J=13.9 Hz, J=3.0 Hz, 1H), 2.74 (td, J=11.7 Hz, J=2.3 Hz, 1H).

### Synthesis of EOB-pyclen oxalate (Compound 6)

1-(chloromethyl)-4-ethoxybenzene (0.34 g, 5 mmol) was added to a solution of pyclen oxalate (0.52 g, 2 mmol) in 30 mL acetonitrile in the presence of K₂CO₃ (0.69 g, 5 mmol). The reaction mixture was refluxed for 3 days, then filtered and concentrated. The crude product was purified by silica gel column chromatography (elution: ethylactate, ethylactate/methanol=9/1, methanol) yielding a yellow oil. Yield: 79%. ¹H NMR (500 MHz, DMSO-d₆). δ ppm: 7.59 (t, J=7.6 Hz, 1H), 7.27 (d, J=8.5 Hz, 2H), 7.18 (d, J=7.6 Hz, 1H), 6.87 (d, J=7.6 Hz, 1H), 6.78 (d, J=8.5 Hz, 2H), 5.33 (d, J=16.3 Hz, 1H), 4.36 (d, J=16.3 Hz, 2H), 4.08 (m, 2H), 3.88 (m, 1H), 3.82 (m, 2H), 3.65 (s, 2H), 3.38-3.42 (m, 2H), 3.01 (m, 1H), 2.82 (m, 1H), 2.79 (m, 1H), 2.65 (m, 1H), 1.33 (t, J=6.9 Hz, 3H).

### Synthesis of EOB-pyclen (Compound 7)

Pyclen oxalate-EOB (0.78 g, 2 mmol) was dissolved in 40 mL MeOH, and 1 mL H₂SO₄ (98%) was slowly added and then refluxed for 24 h. After cooling to room temperature, the solvent was evaporated. 20 mL water was added and pH was adjusted to 7 using K₂CO₃. Water was evaporated and the residue was taken up in dichloromethane. Magnesium sulfate was added and the organic layer was filtered and concentrated. The crude product was purified by column chromatography using neutral alumina (CH₂Cl₂/MeOH: 98/2 to 95/5) yielding a yellow oil. Yield: 53%. ¹H NMR (500 MHz, DMSO-d₆). δ ppm: 7.54 (t, J=7.7 Hz, 1H), 7.19 (d, J=8.5 Hz, 2H) 7.07 (d, J=7.7 Hz, 1H), 6.87 (d, J=7.5 Hz, 1H), 6.74 (d, J=8.5 Hz, 2H), 4.03 (s, 2H), 3.95 (m, 2H), 3.83 (m, 4H), 3.15 (m, 2H), 3.05 (m, 2H), 3.01 (m, 2H), 2.95 (m, 2H), 1.35 (t, J=6.9 Hz, 3H).

### Synthesis of EOB-pyclen-bisacetic acid (EOB-PC2A) (Compound 9)

A solution of tert-butyl bromoacetate (0.47 g, 4.09 mmol) in 25 mL acetonitrile was added to a mixture of EOB-pyclen (0.34 g, 1 mmol) and K₂CO₃ (0.34 g, 2.5 mmol) in 25 mL acetonitrile. The reaction mixture was stirred for two days at room temperature. The solvent was evaporated and the residue was dissolved in dichloromethane, filtered and concentrated. The synthesis of EOB-PC2A bisbutyl ester in the reaction mixture was verified by MALDI-TOF mass spectrometry, m/z (M+1, calcd.): 569.36, m/z (M+1, obsd.): 569.18. The residue was then dissolved in TFA:CH₂Cl₂ (1:2) and stirred for 5 h at room temperature. The solvent was removed by rotavapor, and the crude product was purified by preparative HPLC. Yield: 32%. ¹H NMR (500 MHz, D₂O). δ ppm: 7.79 (t, J=7.5 Hz, 1H), 7.42 (d, J=8.5 Hz, 2H) 7.33 (d, J=7.5 Hz, 1H), 7.18 (d, J=7.5 Hz, 1H), 6.89 (d, J=8.5 Hz, 2H), 4.58 (s, 2H), 3.95 (m, 4H), 3.47-3.63 (m, 4H), 3.36 (s, 2H), 2.67-3.2 (m, 8H), 1.26 (t, J=6.7 Hz, 3H). MALDI-TOF mass spectrum m/z (M−1, calcd.): 455.24, m/z (M−1, obsd.): 455.33.

### Synthesis of Mn(EOB-PC2A) (Compound 10)

EOB-PC2A (45 mg) and 3 equivalent MnCl₂ was dissolved 10 mL deionized water. NaOH aqueous solution (0.2 M) was used to adjusted pH 6.5. The reaction was stirred at room temperature for 24 h. The crude product was purified by preparative HPLC (Eluent A was 10 mm CH₃COONH₄/H₂O, B was acetonitrile and flow rate was 4.0 mL/min, HPLC method A: 0-5 min 100% A, 5-35 min 50% A, 35-40 min 0% A, 40-41 min 100% A, 41-45 min 100% A). The purity of the final product was analyzed by analytical HPLC (Eluent A was 10 mm CH₃COONH₄/H₂O, B was acetonitrile and flow rate was 4.0 mL/min, HPLC method B: 0-5 min 100% A, 5-20 min 50% A, 20-25 min 0% A, 25-30 min 100% A, 30-35 min 100% A). ESI mass spectrum, m/z (M+1, calcd.): 510.16, m/z (M+Na⁺, calcd.): 532.15; m/z (obsd.): 510 (M+1), 532 [M+Na⁺].

### Relaxivity Measurements

Solutions of Mn(EOB-PC2A) were prepared in DPBS or saline containing 4.5% human serum albumin. The T₁ and T₂ relaxation times at 1.5T were measured using a Bruker Minispec Relaxometer (60 Hz, 37° C.; Bruker, Billerica, MA, USA). The T₁ relaxation times were also measured at 3T using an MRS3000 3T small animal scanner from MR solutions (MR Solutions, Surrey, UK) with a T₁ mapping sequence (T_{R}=10 ms, T_{E}=4 ms, T₁=50 ms, FOV=40×40 mm, slice thickness=2 mm, number of slices=1, Nₐᵥ=1, matrix=256×128, flip angle=8°). The r₁ and r₂ relaxivities were calculated as the slope of the 1/T₁ and 1/T₂ vs. Mn concentration, respectively.

### Cell Culture

DU145 cells were purchased from American Type Culture Collection (ATCC, Manassas, VA, USA). HepG2 cells were kindly gifted by Dr. Zhenghong Lee at Case Western Reserve University (CWRU, Cleveland, OH, USA). All cell lines were cultured in Dulbecco's Modified Eagle's Medium (DMEM, Gibco, Amarillo, TX, USA) supplemented with 1% penicillin-streptomycin (Gibco) and 10% fetal bovine serum (Gibco) and incubated at 37° C. and 5% CO₂.

### Cell Viability Analysis

Cells in culture were trypsonized and counted. In a 96-well plate, cells were seeded at 5,000 cells per well and incubated for 1 day to adhere and proliferate. Cells were then incubated with increasing concentrations of the liver-targeted MBCA for 48 hours. To measure cell viability, a CCK8 assay was performed according to the manufacturer's instructions (Dojindo Molecular Technologies, Inc., Kumamoto, JP). Percent cell viability was calculated as the signal intensity of the treated well divided by the signal intensity of the untreated control wells after accounting for background signal.

### Cell Uptake Analysis

Cells were plated in a 6-well plate and incubated for 1 day to adhere and proliferate. Cells were then incubated for 2 hours with 2 mM Mn(EOB-PC2A), after which they were washed three times with DPBS, trypsonized, and pelleted. Cells were resuspended in 250 μL of 75% HNO₃ for 48 hours for dissolution, after which 4.75 mL distilled water was added. The samples were centrifuged at 4,000 rpm for 30 minutes to pellet any non-dissolved components and the supernatant was collected. Manganese content in the cell solution was quantified by inductively coupled plasma optical emission spectroscopy (ICP-OES 730, Agilent Technologies, Santa Clara, CA, USA).

### Animals

All animal and imaging procedures were performed according to an approved Institutional Animal Care and Use Committee protocol. Male C57BL6 mice were purchased from Jackson Labs (Bar Harbor, ME, USA), housed in an animal facility.

### Magnetic Resonance Imaging

All imaging experiments were performed using the MRS3000 3T small animal scanner. Mice (n=5) were anesthetized with 2% isoflurane, the tail vein was cannulized with a catheter, followed by transferal to the heated mouse bed of the MRI scanner. Mice were monitored with a respiration monitor while in the scanner. Before and at 15 and 30 minutes after administration of 60 μmol/kg Mn(EOB-PC2A), mice were imaged with a T₁w axial fast spin-echo sequence with respiratory gating (T_{R}=305 ms, T_{E}=11 ms, FOV=40×40 mm, slice thickness=1 mm, number of slices=15, Nₐᵥ=4, matrix=256×248) and a 3D FLASH sequence (T_{R}=16 ms, T_{E}=0 ms, T₁=1000 ms, FOV=70×35 mm, slice thickness=35 mm, number of slices=1, Nₐᵥ=3, matrix=256×128, flip angle=25°). Mice underwent an additional T₁w axial spin-echo and 3D FLASH acquisitions at 24 hours post-injection.

### MR Image Analysis

MR images were exported in DICOM format, and processed and analyzed using FIJI software (https://imagej.net/software/fiji/). The signal-to-noise ratio [sₜᵢₛₛᵤₑ/σₙₒᵢₛₑ] and contrast-to-noise ratio [(sₜᵢₛₛᵤₑ−s_{muscle})/σₙₒᵢₛₑ] were calculated for the relevant tissues of each mouse at every time point. Subtraction images were produced for the 3D FLASH images by first dividing the pixel values of the images of each acquisition by the scaling factor assigned by the MRS software, and then subtracting the corresponding pre-contrast baseline images from the post-contrast images. Maximum intensity projections and 3-dimensional projections were produced for the raw and subtraction 3D FLASH images in FIJI using the “Z-project” function and the “3D-project” function with interpolation, respectively.

### Statistical Analysis

All experiments were performed in triplicate unless otherwise specified. For mouse experiments, relevant measurements and values (i.e., SNR or CNR) at the different acquisition time points were paired by mouse. For the remainder of experiments, samples were unpaired. For experiments analyzing one independent variable with two groups, a paired t-test was used. For experiments analyzing one independent variable with more than two groups, a one-way ANOVA analysis was performed followed by Tukey-Kramer post-hoc testing. For experiments analyzing two independent variables, a two-way ANOVA analysis was performed followed by Tukey-Kramer post-hoc testing. P-values less than 0.05 were considered statistically significant (*, p<0.05; **, p<0.01; ***, p<0.001; ****, p<0.0001). All quantitative data is presented as mean±standard deviation.

### Results

The synthetic route of Mn(EOB-PC2A) is shown in FIG. 1. Pyclen was first synthesized and then reacted with diethyloxalate in methanol to produce pyclen oxalate with one reactive secondary amine group for functionalization. EOB-pyclen was synthesized by reacting pyclen oxalate with 1-chloromethyl-4-ethoxybenzene in the presence of K₂CO₃, followed by deprotection with sulfuric acid. The chelating ligand EOB-PC2A was synthesized by reaction of EOB-pyclen with tert-butyl-bromoacetate followed by deprotection in TFA:DCM (1:2). The MRI contrast agent Mn(EOB-PC2A) was finally prepared by reacting EOB-PC2A with MnCl₂ in water at a pH of 6.5. The ligand EOB-PC2A was characterized by ¹H-NMR and MALDI-TOF mass spectrometry. The contrast agent Mn(EOB-PC2A) was characterized by ESI mass spectrometry and its purity was determined by HPLC.

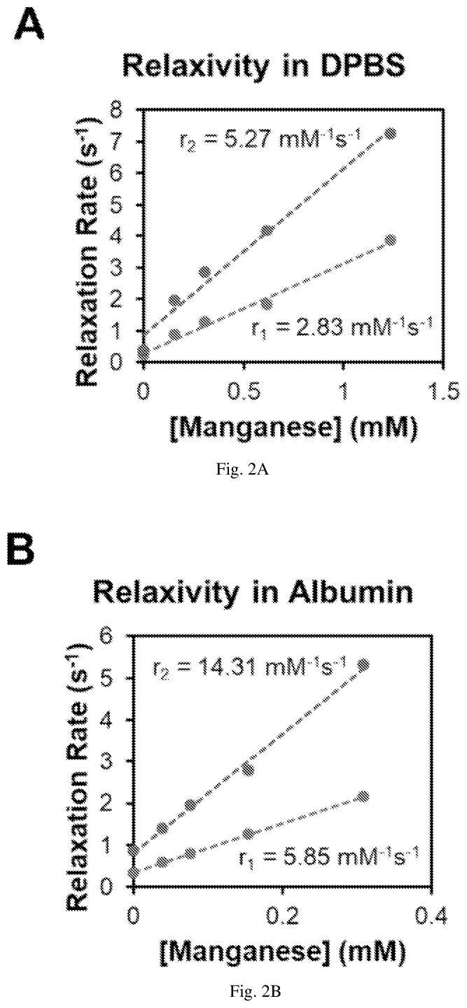

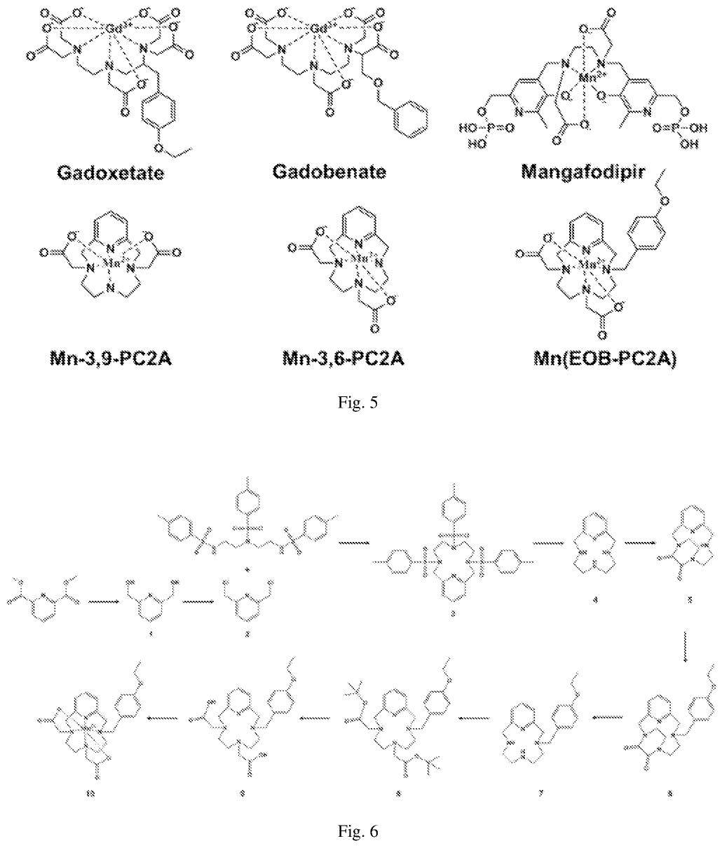

For comparison with relevant contrast agents, the r₁ and r₂ relaxivities of Mn(EOB-PC2A) were calculated based on linear estimation of relaxation rate versus concentration. At 1.5 T in DPBS, Mn(EOB-PC2A) has r₁ and r₂ relaxivities of 2.83 mM⁻¹s⁻¹ and 5.27 mM⁻¹s⁻¹, respectively, which are higher than the relaxivities reported for mangafodipir (r1, _{mangafodipir}=1.6 mM⁻¹s⁻¹, r_{2, mangafodipir}=2.1 mM⁻¹s⁻¹), and are comparable but slightly higher than those reported for untargeted Mn(II) pyclen diacetates in water (r_{1, Mn-3,6-PC2A}=2.4 mM⁻¹s⁻¹, r_{2, Mn-3,6-PC2A}=4.2 mM⁻¹s⁻¹; r_{1, Mn-3,9-PC2A}=2.2 mM⁻¹s⁻¹, r_{2, Mn-3,9-PC2A}=4.8 mM⁻¹s⁻¹) (FIG. 2A, Table 1). In addition, the r₁ relaxivity of Mn(EOB-PC2A) in DPBS at 3 T is 2.61 mM⁻¹s⁻¹, slightly lower than that at 1.5T (FIG. 7). The r₁ and r₂ relaxivities of Mn(EOB-PC2A) at 1.5T increase to 5.85 mM⁻¹s⁻¹ and 14.31 mM⁻¹s⁻¹, respectively, in 4.5% human serum albumin (HSA) (FIG. 2B, Table 1). The r₁ relaxivity of Mn(EOB-PC2A) in 4.5% HSA is comparable to but slightly lower than the liver-targeted GBCAs in human plasma (r_{1, gadoxetate}=6.9 mM⁻¹s⁻¹; r_{1, gadobenate}=6.3 mM⁻¹s⁻¹), suggesting its potential for similar performance in T₁-weighted acquisitions with a non-Gd(III) contrast agent.²² Additionally, the r₁ relaxivity of Mn(EOB-PC2A) in 4.5% HSA represents a stark increase over mangafodipir in human plasma (r_{1, mangafodipir}=3.6 mM⁻¹s⁻¹) and untargeted Mn(II) pyclen diacetates in Seronorm (r_{1, Mn3,6-PC2A}=3.0 mM⁻¹s⁻¹; r_{1, Mn3,9-PC2A}=3.3 mM⁻¹s⁻¹) (Table 1). The substantial relaxivity increase of Mn(EOB-PC2A) relative to the untargeted Mn(II) pyclen diacetates is due in large part to the addition of the EOB ligand, the nonpolar nature of which facilitates interactions with HSA and allows increased labile water exchange. Furthermore, the r₂ relaxivity of Mn(EOB-PC2A) in 4.5% HSA is substantially higher than any of the liver-targeted GBCAs (r_{2, gadoxetate}=8.7 mM⁻¹s⁻¹; r_{2, gadobenate}=8.7 mM⁻¹s⁻¹) or the MBCAs (r_{2, mangafodipir}=7.1 mM⁻¹s⁻¹; r_{2, Mn-3,6-PC2A}=5.0 mM⁻¹s⁻¹; r_{2, Mn-3,9-PC2A}=6.3 mM⁻¹s⁻¹), suggesting a much stronger T₂ effect for potential use in T₂-weighted acquisitions (Table 1).

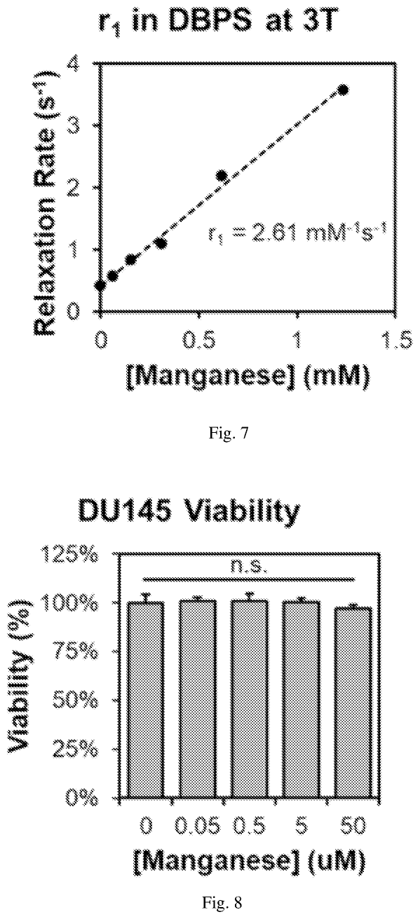

The EOB ligand directly targets OATP1, a transporter that is present on the surface of hepatocytes. To demonstrate the uptake of Mn(EOB-PC2A), HepG2 hepatoma cells, which are commonly used as a proxy cell line for primary liver cells, were incubated with 2 mM Mn(EOB-PC2A) for 2 hours. The HepG2 cells incubated with the agent showed 17 times higher intracellular concentration of Mn(II) than untreated cells, demonstrating the uptake of the agent by liver cells (FIG. 2D). The cytotoxicity of Mn(EOB-PC2A) was assessed by incubation with HepG2 cells and DU145 prostate cancer cells. Both cell lines demonstrated no cytotoxicity at physiologically relevant concentrations of Mn(EOB-PC2A), suggesting a good safety profile at the cellular level for the agent (FIG. 2C, FIG. 8).

TABLE 1

| | | Water/DPBS | | Plasma/Serum | | |
|---|---|---|---|---|---|---|
| | Bₒ | r₁ | r₂ | r₁ | r₂ | Plasma |
| Contrast Agent | (T) | (mM⁻¹ s⁻¹) | | (mM⁻¹ s⁻¹) | | component |
| Mn(EOB-PC2A) | 1.5 | 2.8 | 5.3 | 5.9 | 14.3 | (Human serum |
| | | | | | | albumin) |
| Gadoxetate | 1.5 | 4.7 | 5.1 | 6.9 | 8.7 | (Blood plasma) |
| Gadobenate | 1.5 | 4.0 | 4.3 | 6.3 | 8.7 | (Blood plasma) |
| Mangafodipir | 1.5 | 1.6 | 2.1 | 3.6 | 7.1 | (Blood plasma) |
| Mn-3,6-PC2A | 1.41 | 2.4 | 4.2 | 3.0 | 5.0 | (Seronorm) |
| Mn-3,9-PC2A | 1.41 | 2.2 | 4.8 | 3.3 | 6.3 | (Seronorm) |

Values of r₁ and r₂ relaxivities for Mn(EOB-PC2A) and other relevant contrast agents in water/DPBS and plasma/serum proteins. Relaxivities for Mn(EOB-PC2A) were calculated using relaxation rate measurements from phantoms prepared in DPBS or 4.5% human serum albumin in saline at 1.5T and 37° C. Relaxivities for the other clinically approved agents and non-targeted pyclen diacetates were pulled from the relevant literature in conditions resembling those used in this study.

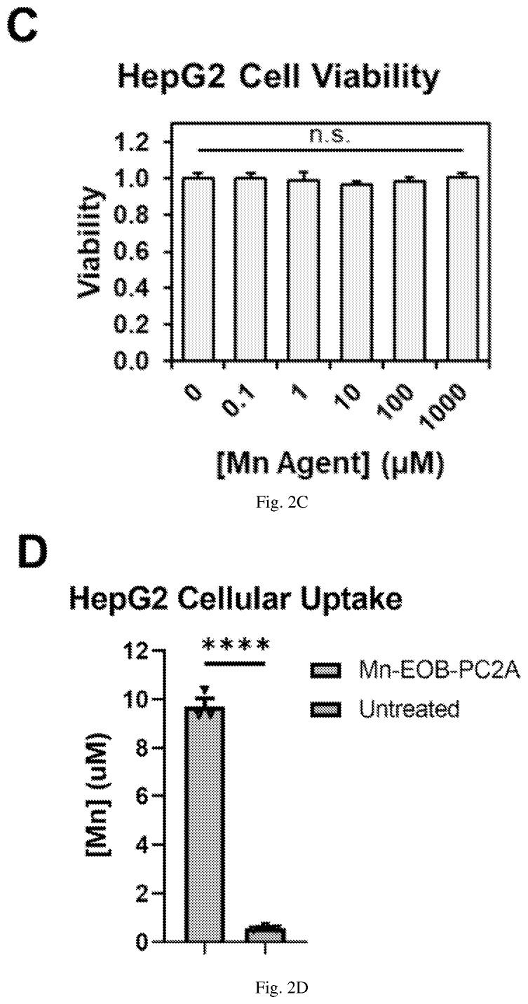

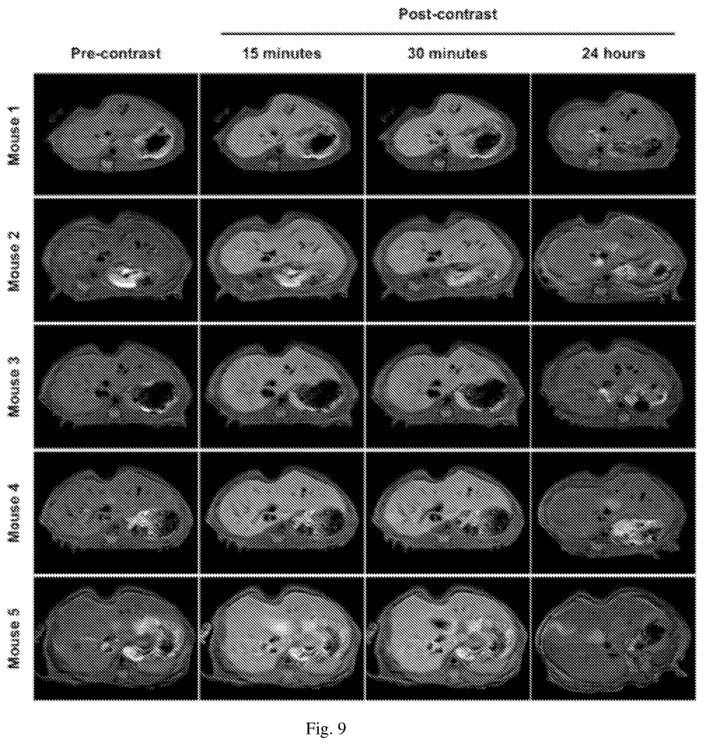

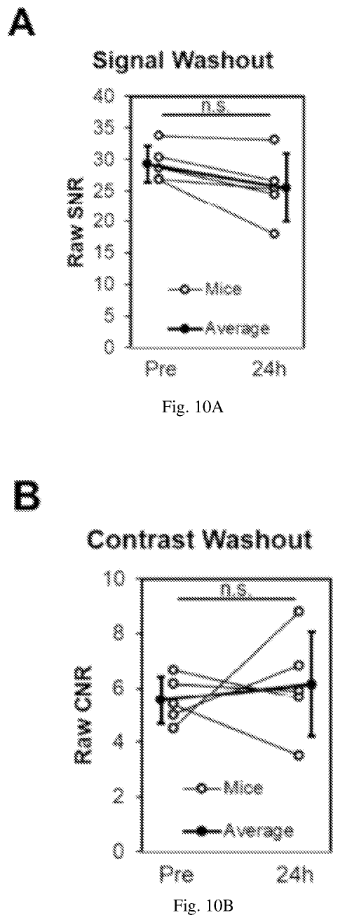

The effectiveness of Mn(EOB-PC2A) for liver-specific contrast enhanced MRI has been determined in C57BL6 mice with T₁-weighted (T₁w) axial fast spin-echo and 3D FLASH sequences (see methods for details). Gadoxetate, the clinically approved gadolinium based contrast agent (GBCA) containing the EOB ligand, exhibits peak enhancement around 20 minutes after administration. Thus, T₁w axial images of the liver and 3D FLASH whole-body images were acquired before, and at 15 minutes, 30 minutes, and 24 hours after intravenous injection of Mn(EOB-PC2A) at a dose of 0.060 mmol/kg. T₁w axial images show substantial enhancement in the liver at both 15 and 30 minutes post-injection (FIG. 3A, FIG. 9). Analysis of MRI signal intensities demonstrates significantly increased signal-to-noise ratio (SNR) and contrast-to-noise ratio (CNR) in the liver at 15 (SNR₁₅ₘ=1.41, p=0.0053; CNR₁₅ₘ=3.20, p=0.0036) and 30 (SNR₃₀ₘ=1.42, p=0.0030; CNR₃₀ₘ=3.12, p=0.0026) minutes post-injection relative to the pre-contrast baseline (FIG. 3B-C). The strong persistent contrast enhancement in the liver at 30 minutes post-injection suggests robust continuous enhancement for imaging, which is consistent with the enhancement kinetics of the liver-targeted GBCAs. After 24 hours, no contrast enhancement is observed in the liver of all tested mice (FIG. 3A, FIG. 9). Analysis of liver SNR (SNR₂₄ₕ=0.86, p=0.055) and CNR (CNR₂₄ₕ=1.16, p=0.6201) at 24 hours demonstrates the return of MR signal to the pre-contrast baseline with no statistically significant difference, suggesting efficient washout of Mn(EOB-PC2A) within 24 hours of injection (FIG. 3D-E, FIG. 10).

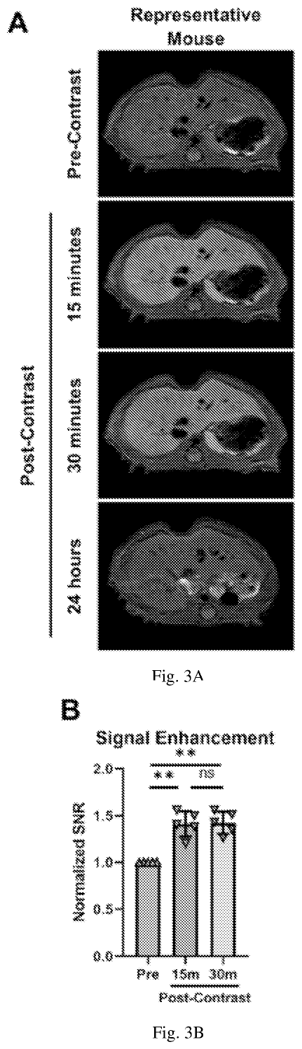

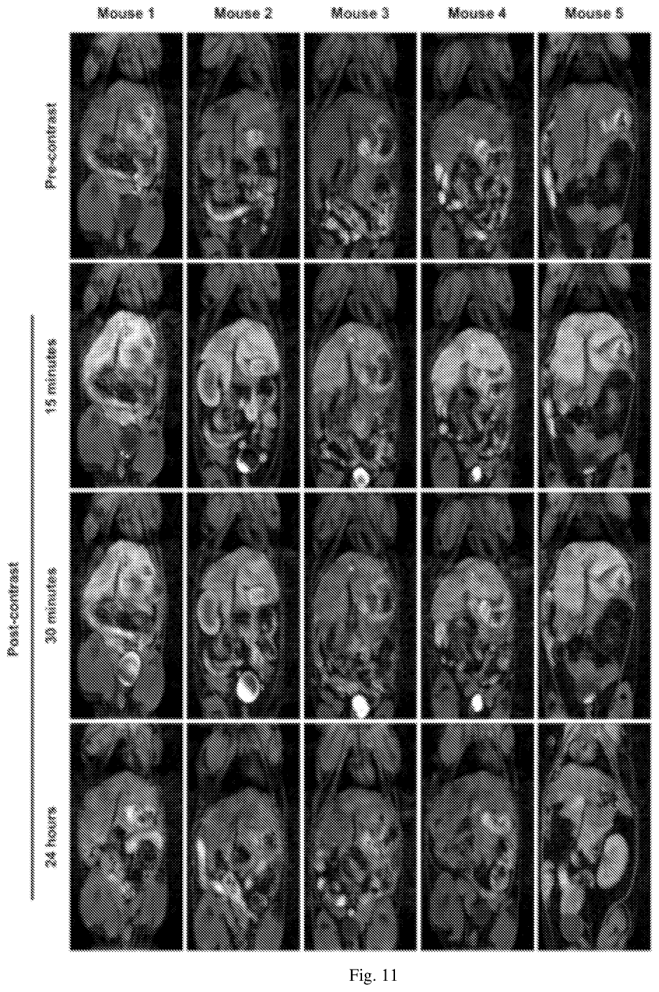

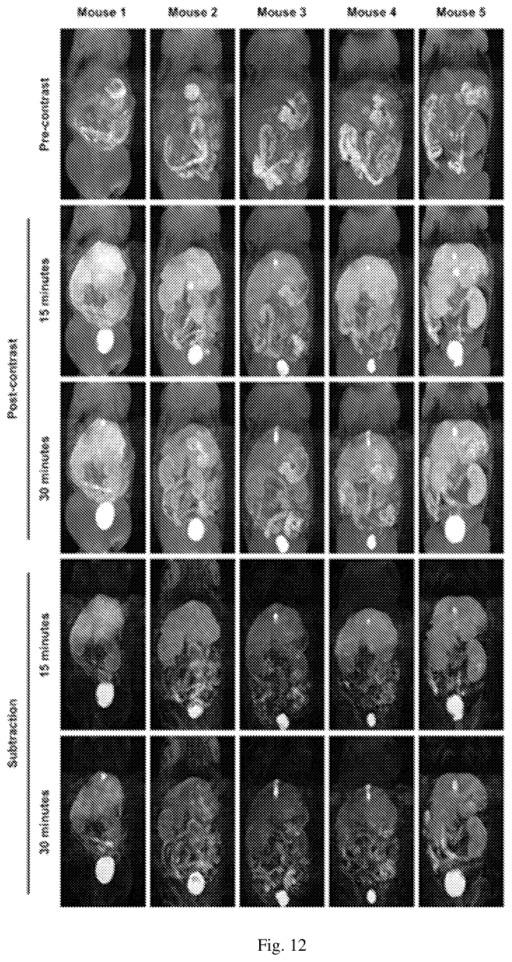

The 3D FLASH whole-body images were used to assess the liver-specificity of Mn(EOB-PC2A) (FIG. 11). FIG. 4A shows the representative maximum intensity projection (MIP) whole-body images before and at different time points post-injection. Signal enhancement is seen in the liver, kidneys, gallbladder, and urinary bladder in the 3D whole body images at 15 and 30 minutes post-injection. The strong signal enhancement and high SNR measurement in the kidneys and urinary bladder suggests that the unbound agent is rapidly excreted via renal filtration (FIG. 4B, Table 2). The strong enhancement in the gallbladder, particularly at 30 minutes post-injection, indicates that the agent is also eliminated via biliary excretion after strong hepatocytic uptake (FIG. 4A, FIG. 12). The elimination mechanisms of Mn(EOB-PC2A) are consistent with the those for EOB-conjugated contrast agents. There is a general lack of enhancement in other off-target tissues and organs (FIG. 4B, FIGS. 11-12, Table 2). Notably, the heart, which often exhibits significant enhancement with MnCl₂ and some reported MBCAs due to the release of free Mn(II), and the highly vascularized lungs showed no significant change in SNR, suggesting a lack of appreciable contrast agent accumulation (FIG. 4B, Table 2). At 24 hours, the whole-body images and SNR analyses showed little to no change in tissue enhancement relative to the pre-contrast baseline (FIG. 4, FIG. 11). The results suggest that Mn(EOB-PC2A) has high liver specificity with little non-specific tissue enhancement.

TABLE 2
Delta SNR values for the 3D FLASH coronal acquisition of various
tissues before and at 15 minutes, 30 minutes, and 24 hours post-injection.
Statistical analysis performed on this data set was done via paired analysis,
hence why the pre-contrast baseline averages all read 0.00 ± 0.00.

| | | Post-Contrast | | |
|---|---|---|---|---|
| Tissue | Pre-Contrast | 15 minutes | 30 minutes | 24 hours |
| Lung | 0.00 ± 0.00 | −0.27 ± 0.44 | −0.41 ± 0.31 | −0.43 ± 0.71 |
| Kidney | 0.00 ± 0.00 | 27.74 ± 7.66 | 21.33 ± 8.28 | 3.87 ± 5.62 |
| Muscle | 0.00 ± 0.00 | −3.63 ± 3.21 | −4.07 ± 5.11 | −6.62 ± 10.13 |
| Liver | 0.00 ± 0.00 | 51.48 ± 17.20 | 29.19 ± 14.28 | 1.36 ± 6.44 |
| Heart | 0.00 ± 0.00 | −0.04 ± 5.85 | −2.28 ± 7.11 | 5.73 ± 16.25 |
| Bladder | 0.00 ± 0.00 | 157.07 ± 39.02 | 199.96 ± 25.86 | −2.58 ± 5.42 |

The EOB-targeted GBCA Gadoxetate has shown great promise in specific liver imaging and comprehensive non-invasive assessment of liver functions with MRI in recent studies. Gadoxetate is effective to assess liver function by direct targeting of the OATP1 transporter and monitoring the uptake and hepatobiliary excretion of the contrast agent. Various quantitative metrics, such as relative liver enhancement, contrast uptake index, hepatic uptake index, liver-spleen index, and functional liver imaging score, have been developed to analyze liver function based on clinical gadoxetate-enhanced MRI. These metrics demonstrated remarkable accuracy at identifying liver fibrosis and cirrhosis as well as predicting hepatic decompensation. This is particularly important from a prognostic standpoint, as patients with decompensated chronic liver diseases are at a much higher risk for liver failure than those with compensated chronic liver diseases. However, due to the safety concerns of releasing toxic free Gd(III) ions from the linear GBCAs, gadoxetate may be limited for repeated clinical assessment of chronic liver diseases. Therefore, Mn(II) based contrast agents have drawn interest as potential replacements for clinical GBCAs. Mn(EOB-PC2A) targets the same OATP1 transporter as gadoxetate and presents a non-gadolinium EOB-targeted macrocyclic alternative for clinical assessment of liver function with MRI. We have shown that Mn(EOB-PC2A) has a comparable r₁ relaxivity and high stability, producing liver-specific enhancement at a relatively low dose. The strong liver uptake of Mn(EOB-PC2A) warrants future assessment of its ability for quantitative assessment of liver function to further evaluate its potential as a direct alternative to gadoxetate.

In conclusion, we have reported the synthesis, characterization, and assessment of a novel EOB-conjugated MBCA Mn(EOB-PC2A) for liver-specific contrast enhanced MRI. Mn(EOB-PC2A) possesses higher r₁ relaxivity than the non-targeted MBCAs, and the EOB ligand enhances interactions with serum albumin to further improve the relaxivity comparable to the liver-specific GBCAs. MRI with Mn(EOB-PC2A) demonstrated long lasting and robust liver-centric contrast enhancement in mice at a clinically relevant dose. Its high stability and low cytotoxicity indicate a good safety profile for clinical use. Our results demonstrate the promise of Mn(EOB-PC2A), and merits continued development as a non-gadolinium alternative for liver-specific MRI.

### Example 2

### Synthesis and Characterization of ZD2-(Mn-PCTA-MA) Conjugates as Non-Gadolinium Targeted Contrast Agents for Cancer MR Molecular Imaging

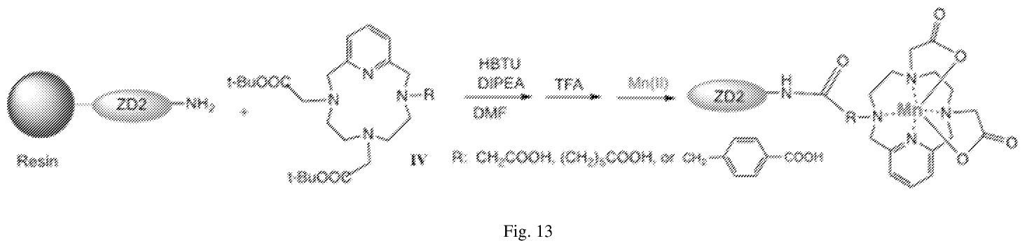

The linear ZD2 peptide is synthesized using solid phase chemistry. The protected PCTA, 3,6-bis-(t-Bu)-PCTA derivatives of different spacers (IV), are synthesized first, using chloroacetic acid, 6-chlorohexanoic acid, or 4-chloromethylbenzoic acid. Different spacers are evaluated for their rigidity on relaxivities to select and develop targeted agents with high relaxivities. The ZD2-targeted MBCAs is synthesized by conjugating compound IV in solid phase as described in FIG. 13. The targeted ligands are cleaved from the resin and deprotected in TFA. After the purification and characterization, the ligands are complexed with an excess of MnCl₂ in water at pH 6.5 and room temperature for 24 h to synthesize the targeted MBCAs. The final products are purified using flash chromatography equipped with a preparative C-18 column and lyophilized to obtain targeted MBCAs with high purity.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A tissue-specific manganese based magnetic resonance imaging (MRI) agent, the agent comprising the formula:
T-L-C
wherein T includes at least one cell specific or tissue specific targeting moiety; C includes at least one pyclen based chelating agent complexed with a Mn ion, and L includes at least one optional linker that covalently links the at least one targeting moiety to the at least one chelating agent.

2: The agent of claim 1, wherein the pyclen based chelating is selected from pyclen diacetate, pyclen triacetate, and a derivative thereof.

3: The agent of claim 1, wherein the pyclen based chelating agent comprises at least one of pyclen-3,9-diacetate, pyclen-3,6-diacetate, or pyclen-3,6,9-triacetate.

4: The agent of claim 1 comprising the formula: 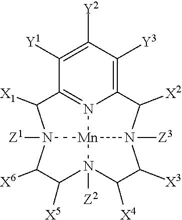
or a pharmaceutically acceptable salt thereof, wherein:
X¹, X², X³, X⁴, X⁵, and X⁶ are each independently H, -L¹-T¹, or -T¹;
Y¹, Y², and Y³ are each independently H, -L¹-T¹, or -T¹;
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is cell specific or tissue specific targeting moiety; and where at least one of X¹, X², X³, X⁴, X⁵, X⁶, Y¹, Y², Y³, Z¹, Z², or Z³ is -L¹-T¹ or -T¹.

5: The agent of claim 1, comprising the formula: 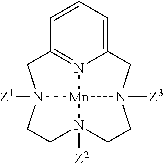
or a pharmaceutically acceptable salt thereof,
wherein:
Z¹, Z², and Z³ are each independently H, —CH₂—C(O)O⁻, -L¹-T¹, or -T¹, where at least two of Z¹, Z², or Z³ is —CH₂—C(O)O⁻;
L¹ is a linker;
T¹ is cell specific or tissue specific targeting moiety; and where at least one of Z¹, Z², or Z³ is -L-T or -T.

6: The agent of claim 1, comprising the formula: 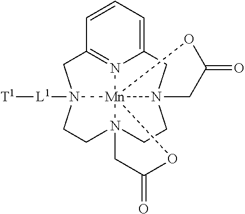
or a pharmaceutically acceptable salt thereof;
wherein L¹ is an optional linker and T¹ is a cell specific or tissue specific targeting moiety.

7: The agent of claim 1, wherein the at least one targeting moiety includes small molecule, peptide, antibody, or aptamer.

8: The agent of claim 1, wherein the targeting moiety binds to hepatocytes and/or liver tissue of a subject.

9: The agent of claim 1, wherein the targeting moiety comprises an ethoxybenzyl group.

10: The agent of claim 9, comprising the following formula: 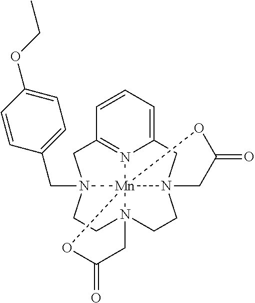
or a pharmaceutically acceptable salt thereof.

11: The agent of claim 1, wherein the targeting moiety binds a cancer cell antigen.

12: The agent of claim 11, wherein the cancer cell antigen comprises at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), extracellular domain B of fibronectin (EDB-FN), extracellular domain A of fibronectin (EDB-FN), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

13: The agent of claim 1, wherein the targeting moiety comprises at least one of a peptide that includes an amino acid sequence that specifically bind to EDB-FN or EDA-FN.

14: The agent of claim 1, wherein the targeting peptide include an amino sequence selected from TVRTSAD (SEQ ID NO: 1), NWGDRIL (SEQ ID NO: 2), NWGKPIK (SEQ ID NO: 3), SGVKSAF (SEQ ID NO: 4), GVKSYNE (SEQ ID NO: 5), IGKTNTL (SEQ ID NO: 6), IGNSNTL (SEQ ID NO: 7), IGNTIPV (SEQ ID NO: 8), and LYANSPF (SEQ ID NO: 9), WNYPFRL (SEQ ID NO: 19), SNTSYVN (SEQ ID NO: 20), SFSYTSG (SEQ ID NO: 21), WSPAPMS (SEQ ID NO: 22), TREHPAQ (SEQ ID NO: 23), or ARIIDNA (SEQ ID NO: 24), cyclic peptides thereof, and retro-inverso amino acid sequences thereof.

15: The agent of claim 1, wherein the targeting moiety is directly linked to the chelating agent.

16: The agent of claim 1, wherein the targeting moiety is linked to the chelating with a linker.

17: The agent of claim 16, wherein the linker comprises a homo- or hetero-bifunctional linker, where two reactive groups are separated by at least one of an alkylene, alkenylene, alkynylene, cycloalkylenes, arylene, or araalkylene, which is optionally substituted with one or more heteroatoms.

18: The agent of claim 16, wherein the linker includes a C₁-C₆ alkylene, C₃-C₆-cycloakylene, 3 to 6 membered heterocyclylene, or arylene.

19: A pharmaceutical composition comprising the tissue-specific manganese based magnetic resonance imaging (MRI) agent of claim 1 in an amount sufficient to provide a desired level of contrast and at least one pharmaceutically acceptable carrier.

20-27. (canceled)
